(19) United States
(12) Patent Application Publication
(10) Pub. No.: US 2014/0257058 A1
(43) Pub. Date: Sep. 11, 2014

(54) AUTOMATED PERSONAL MEDICAL DIAGNOSTIC SYSTEM, METHOD, AND ARRANGEMENT

(71) Applicant: SCANADU INCORPORATED, MOFFETT FIELD, CA (US)

(72) Inventors: Ivo Clarysse, San Francisco, CA (US); Misha Chellam, San Francisco, CA (US); Walter Debrouwer, Uccle (BE); Anthony Smart, Costa Mesa, CA (US)

(21) Appl. No.: 14/350,798

(22) PCT Filed: Oct. 19, 2012
(86) PCT No.: PCT/US12/61046

§ 371 Date: Apr. 9, 2014

Related U.S. Application Data
(60) Provisional application No. 61/549,134, filed on Oct. 19, 2011.

(51) Int. Cl. A61B 5/00

(52) U.S. Cl. CPC A61B 5/0022
USPC 600/301

(57) ABSTRACT

An automated personal medical diagnostic system and arrangement, including: at least one sensor configured to measure and/or sense at least one physiological condition and generate or acquire sensor data; at least one computing device configured to process at least a portion of the sensor data and generate diagnostic data based at least partially on the sensor data; and at least one user interface configured for user interaction; wherein the diagnostic data at least partially comprises at least one of the following: indicator data, medical diagnostic data, trigger data, or any combination thereof. A method for automated medical diagnosis is also disclosed.

### CROSS REFERENCE TO RELATED APPLICATIONS

This application claims benefit of priority from U.S. Provisional Patent Application No. 61/549,134, filed Oct. 19, 2011, which is incorporated by reference in its entirety.

### BACKGROUND OF THE INVENTION

1. Field of the Invention

The present invention relates generally to mobile computing and communication devices, medical measurement sensors, and medical diagnostic systems, and in particular to an automated personal medical diagnostic system, method, and arrangement.

2. Description of the Related Art

In the field of healthcare, the primary goals are the determination of any conditions or issues relating to a patient's health, and providing a diagnosis or other assessment in order to address the needs of the patient. As is well known, and in a traditional healthcare setting, the patient visits the doctor and describes his or her symptoms. In many instances, further testing is required, such as through a visit to a hospital, some healthcare services provider, and/or using one or more personal sensors. For example, the patient may wear a variety of sensors or otherwise be associated with such sensors in order to measure and/or sense a specified physiological condition. Finally, based upon the input from the patient and the measurement data from the sensors, the doctor or other clinician can perform the necessary analytics and provide information regarding the patient's diagnosis. Of course, this diagnosis can include recommendations for care or treatment, or may require additional investigation, such as through continued use of one or more sensors.

While the above-described known healthcare process is used throughout the world—as technology advances, so does the ability to use this technology to provide faster and more effective health care to the patient. Accordingly, various systems and arrangements exist that provide applications and networks that offer mobile (and other) computing systems for patient data gathering and analysis. For example, various healthcare-related systems and environments are shown and described in one or more of the following: U.S. Patent Publication Nos.: 2010/0287001; 2010/0056880; 2010/0049006; 2009/0097623; 2009/0273467; 2009/0259494; and 2005/0204310; and U.S. Pat. Nos. 7,905,832; 7,616,110; 5,701,904; 7,515,043; 7,215,991; 5,701,904; and 5,935,060.

Given the importance of improving the efficiency and effectiveness of health and patient care, there remains considerable room in the art for providing improved medical and diagnostic systems, such as improved automated personal medical diagnostic systems, methods, and arrangements.

### SUMMARY OF THE INVENTION

Generally, provided is an automated personal medical diagnostic system, method, and arrangement that address or overcome certain drawbacks and/or deficiencies in existing patient and healthcare systems, such as those systems implemented in a networked environment. Preferably, provided is an automated personal medical diagnostic system, method, and arrangement that allows for the local measurement of one or more physiological conditions in a patient. Preferably, provided is an automated personal medical diagnostic system, method, and arrangement that provides for the provision, e.g., local display, of diagnostic data to at least one user, such as the patient.

Accordingly, and in one preferred and non-limiting embodiment, provided is an automated personal medical diagnostic system, including: at least one sensor configured to measure and/or sense at least one physiological condition and generate or acquire sensor data; at least one computing device configured to process at least a portion of the sensor data and generate diagnostic data based at least partially on the sensor data; and at least one user interface configured for user interaction. Further, the diagnostic data at least partially includes at least one of the following: indicator data, medical diagnostic data, trigger data, or any combination thereof.

In another preferred and non-limiting embodiment, provided is a method for automated medical diagnosis. This method includes: associating with at least one user at least one sensor configured to measure and/or sense at least one physiological condition and generate or acquire sensor data; providing at least one computing device configured to process at least a portion of the sensor data and generate diagnostic data based at least partially on the sensor data; and providing at least one user interface configured for user interaction. The diagnostic data at least partially includes medical diagnostic data, and the medical diagnostic data at least partially includes at least one of the following: a condition, a potential condition, an elimination of a potential condition, an analysis, a severity of a condition, a severity of a potential condition, a relevancy of a condition, a recommendation, a recommendation for further analysis, a recommendation for further monitoring, a recommendation for medical consultation, a recommendation for tele-medical consultation, a recommendation for an in-person consultation, a recommendation for urgent care, a recommendation for emergency care, a recommendation for further assistance, a recommendation including further information, a recommendation including health data, a recommendation including wellness data, or any combination thereof.

In another preferred and non-limiting embodiment, provided is an automated personal medical diagnosis system, including: a user interface hosted on a mobile communication platform with a display, wherein the user interface is configured to accept input from one or more users via a graphical interface shown on the display; and one or more devices including a processing unit, one or more sensors, and a wireless network interface, wherein the processing unit is configured to process the input from the one or more users and the one or more sensors in accordance with one or more algorithms, and wherein the one or more devices and the one or more wireless network interfaces are configured to enable wireless communication between the one or more devices and the mobile communication platform.

In a still further preferred and non-limiting embodiment, provided is a method for automated medical diagnosis, including: providing a mobile medical diagnosis system including one or more processors, one or more sensors and a memory, wherein the system is configured to store one or more medical diagnosis algorithms within the memory; accepting, at the mobile medical diagnosis system, user input and information from the one or more sensors; and providing, from the medical diagnosis system, one or more medical diagnoses, wherein the medical diagnoses are generated using the one or more processors of the mobile medical diagnosis system based on the user input and the information from the one or more sensors in accordance with the one or more medical diagnosis algorithms.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a schematic view of one embodiment of an automated personal medical diagnostic system and arrangement according to the principles of the present invention;

FIG. 2 is a schematic view of another embodiment of an automated personal medical diagnostic system and arrangement according to the principles of the present invention;

FIG. 3 is a schematic view of one embodiment of certain hardware components for use in an automated personal medical diagnostic system and arrangement according to the principles of the present invention;

FIG. 4 is a schematic view of one embodiment of certain software components for use in an automated personal medical diagnostic system and arrangement according to the principles of the present invention;

FIG. 5 is a schematic view of one embodiment of certain software modules for use in an automated personal medical diagnostic system and arrangement according to the principles of the present invention;

FIG. 6 is a schematic view of one embodiment of certain hardware connections and sensors for use in an automated personal medical diagnostic system and arrangement according to the principles of the present invention;

FIG. 7 is a schematic view of another embodiment of an automated personal medical diagnostic system and arrangement according to the principles of the present invention;

FIG. 8 is a schematic view of another embodiment of certain hardware components for use in an automated personal medical diagnostic system and arrangement according to the principles of the present invention;

FIG. 9 is a schematic view of another embodiment of certain software components for use in an automated personal medical diagnostic system and arrangement according to the principles of the present invention; and

FIG. 10 is a schematic view of a computer and network infrastructure according to the prior art.

### DETAILED DESCRIPTION OF THE PREFERRED EMBODIMENTS

For purposes of the description hereinafter, the terms “end”, “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that may be wired and/or wireless in nature. Additionally, two units or components may be in communication with each other even though the data transmitted may be modified, processed, and/or routed between the first and second unit or component. For example, a first unit may be in communication with a second unit even though the first unit passively receives data, and does not actively transmit data to the second unit. As another example, a first unit may be in communication with a second unit if an intermediary unit processes data from one unit and transmits processed data to the second unit. It will be appreciated that numerous other arrangements are possible. The components or units may be directly connected to each other or may be connected through one or more other devices or components. The various coupling components for the devices can include but are not limited to the Internet, a wireless network, a conventional wire cable, an optical cable or connection through air, water or any other medium that conducts signals, and any other coupling device or medium.

Generally, and in various preferred and non-limiting embodiments, the invention provides systems and methods for acquiring, evaluating, screening, and/or presenting medical diagnosis to a user. For example, in certain preferred and non-limiting embodiments, provided are systems and methods for performing an automated medical diagnosis using a mobile device or communication platform, such as a smartphone. Various aspects of the invention described herein may be applied to any of the particular applications set forth below or in any other type of medical analytical/diagnostic setting. Further, the invention may be applied as a stand-alone method or system, or as part of an integrated medical diagnostic system. It should be understood that different aspects of the invention can be appreciated individually, collectively, or in combination with each other.

Hereinafter, this invention is described in terms of functional block components, optional selections, and various processing steps. Such functional blocks may be realized by any number of hardware and/or software components configured to perform to specified functions. For example, the invention may employ various integrated circuit components (e.g., memory elements, processing elements, logic elements, lookup tables, and the like), which may carry out a variety of functions under the control of one or more microprocessors or other control devices. Similarly, the software components of this invention may be implemented with any programming or scripting languages such as C, C#, C++, Java, assembler, extensible markup language (XML), extensible stylesheet transformations (XSLT), with the various algorithms being implemented with any combination of data structures, objects, processes, routines, or other programming elements.

Further, it should be noted that this invention may employ any number of conventional techniques for data transmission, signaling, data processing, network control, and the like. In addition, many applications of the present invention could be formulated. The exemplary network disclosed herein may include any system for exchanging data or transacting business, such as the Internet, an intranet, an extranet, WAN, LAN, satellite or cellular communication networks, and/or the like. The terms “Internet” or “network”, as used herein, may refer to the Internet, any replacement, competitor or successor to the Internet, or any public or private internetwork, intranet or extranet that is based upon open or proprietary protocols. Specific information related to the protocols, standards, and application software used in connection with the Internet may not be discussed herein.

Where required, a system user may interact with the system to complete a transaction via any input device or user interface, such as presses or gestures on a touchscreen, user U or patient actions that cause a change in readings obtained from sensors, keypad presses, and so on. Similarly, this invention could be used with any kind of smartphone (e.g., Apple iPhone, BlackBerry), handheld computer (e.g., Apple iPad) or used with any type of personal computer, network computer, workstation, minicomputer, mainframe or the like running any operating system, such as any version of Android, Linux, Windows, Windows NT, Windows 2000, Windows XP, MacOS, UNIX, Solaris, iOS or the like. The invention could be implemented using one or more of the following communication protocols: TCP/IP, X.25, SNA, AppleTalk, SCSI, NetBIOS, OSI, GSM, or any number of communication protocols. Moreover, the system contemplates the use, sale, or distribution of any goods, services, or information over any network having similar functionality described herein.

A variety of conventional communications media and protocols may be used for the data links. For example, data links may be an Internet Service Provider (ISP) configured to facilitate communications over a local loop as is typically used in connection with standard modern communication, cable modem, dish networks, ISDN, DSL lines, GSM, G4/LTE, WDMCA, or any wireless communication media.

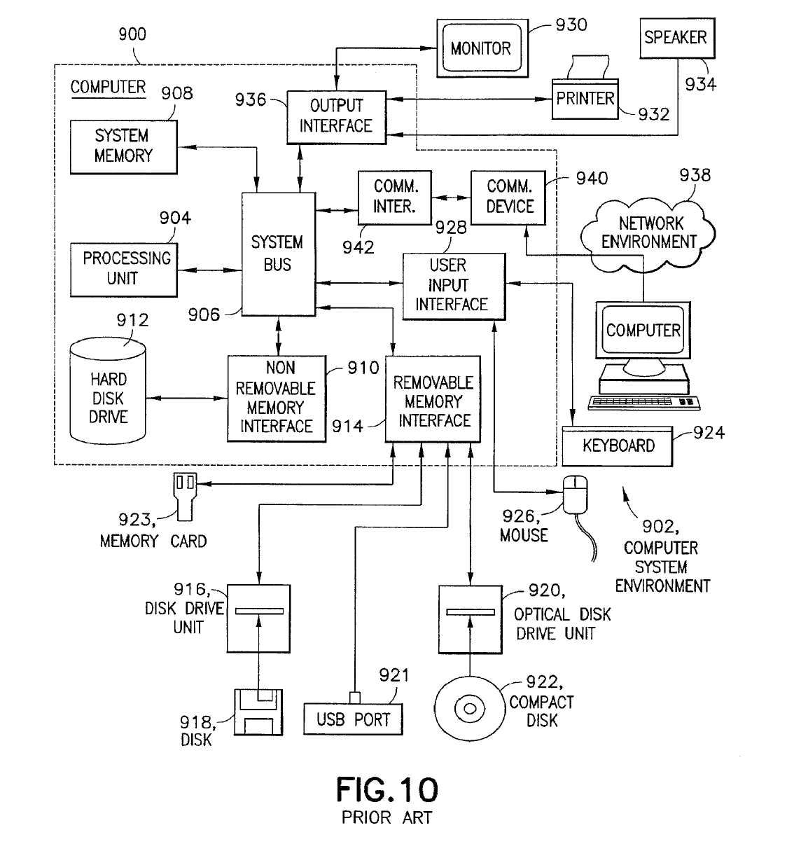

Still further, and discussed hereinafter, it is to be recognized that some or all of the functions, aspects, features, and instances of the present invention may be implemented on a variety of computing devices and systems, wherein these computing devices include the appropriate processing mechanisms and computer-readable media for storing and executing computer-readable instructions, such as programming instructions, code, and the like. As shown in FIG. 10, personal computers 900, 944, in a computing system environment 902 are provided. This computing system environment 902 may include, but is not limited to, at least one computer 900 having certain components for appropriate operation, execution of code, and creation and communication of data. For example, the computer 900 includes a processing unit 904 (typically referred to as a central processing unit or CPU) that serves to execute computer-based instructions received in the appropriate data form and format. Further, this processing unit 904 may be in the form of multiple processors executing code in series, in parallel, or in any other manner for appropriate implementation of the computer-based instructions.

In order to facilitate appropriate data communication and processing information between the various components of the computer 900, a system bus 906 is used. The system bus 906 may be any of several types of bus structures, including a memory bus or memory controller, a peripheral bus, or a local bus using any of a variety of bus architectures. In particular, the system bus 906 facilitates data and information communication between the various components (whether internal or external to the computer 900) through a variety of interfaces, as discussed hereinafter.

The computer 900 may include a variety of discrete computer-readable media components. For example, this computer-readable media may include any media that can be accessed by the computer 900, such as volatile media, non-volatile media, removable media, non-removable media, etc. As a further example, this computer-readable media may include computer storage media, such as media implemented in any method or technology for storage of information, such as computer-readable instructions, data structures, program modules, or other data, random access memory (RAM), read only memory (ROM), electrically erasable programmable read only memory (EEPROM), flash memory, or other memory technology, CD-ROM, digital versatile disks (DVDs), or other optical disk storage, magnetic cassettes, magnetic tape, magnetic disk storage, or other magnetic storage devices, or any other medium which can be used to store the desired information and which can be accessed by the computer 900. Further, this computer-readable media may include communications media, such as computer-readable instructions, data structures, program modules, or other data in other transport mechanisms and include any information delivery media, wired media (such as a wired network and a direct-wired connection), and wireless media. Computer-readable media may include all machine-readable media. Of course, combinations of any of the above should also be included within the scope of computer-readable media.

The computer 900 further includes a system memory 908 with computer storage media in the form of volatile and non-volatile memory, such as ROM and RAM. A basic input/output system (BIOS) with appropriate computer-based routines assists in transferring information between components within the computer 900 and is normally stored in ROM. The RAM portion of the system memory 908 typically contains data and program modules that are immediately accessible to or presently being operated on by processing unit 904, e.g., an operating system, application programming interfaces, application programs, program modules, program data, and other instruction-based computer-readable codes.

With continued reference to FIG. 10, the computer 900 may also include other removable or non-removable, volatile or non-volatile computer storage media products. For example, the computer 900 may include a non-removable memory interface 910 that communicates with and controls a hard disk drive 912, i.e., a non-removable, non-volatile magnetic medium; and a removable, non-volatile memory interface 914 that communicates with and controls a magnetic disk drive unit 916 (which reads from and writes to a removable, non-volatile magnetic disk 918), an optical disk drive unit 920 (which reads from and writes to a removable, non-volatile optical disk 922, such as a CD ROM), a Universal Serial Bus (USB) port 921 for use in connection with a removable memory card, etc. However, it is envisioned that other removable or non-removable, volatile or non-volatile computer storage media can be used in the exemplary computing system environment 900, including, but not limited to, magnetic tape cassettes, DVDs, digital video tape, solid state RAM, solid state ROM, etc. These various removable or non-removable, volatile or non-volatile magnetic media are in communication with the processing unit 904 and other components of the computer 900 via the system bus 906. The drives and their associated computer storage media discussed above and illustrated in FIG. 10 provide storage of operating systems, computer-readable instructions, application programs, data structures, program modules, program data, and other instruction-based computer-readable code for the computer 900 (whether duplicative or not of this information and data in the system memory 908).

A user may enter commands, information, and data into the computer 900 through certain attachable or operable input devices, such as a keyboard 924, a mouse 926, etc., via a user input interface 928. Of course, a variety of such input devices may be used, e.g., a microphone, a trackball, a joystick, a touchpad, a touch-screen, a scanner, etc., including any arrangement that facilitates the input of data, and information to the computer 900 from an outside source. As discussed, these and other input devices are often connected to the processing unit 904 through the user input interface 928 coupled to the system bus 906, but may be connected by other interface and bus structures, such as a parallel port, game port, or a universal serial bus (USB). Still further, data and information can be presented or provided to a user in an intelligible form or format through certain output devices, such as a monitor 930 (to visually display this information and data in electronic form), a printer 932 (to physically display this information and data in print form), a speaker 934 (to audibly present this information and data in audible form), etc. All of these devices are in communication with the computer 900 through an output interface 936 coupled to the system bus 906. It is envisioned that any such peripheral output devices be used to provide information and data to the user.

The computer 900 may operate in a network environment 938 through the use of a communications device 940, which is integral to the computer or remote therefrom. This communications device 940 is operable by and in communication with the other components of the computer 900 through a communications interface 942. Using such an arrangement, the computer 900 may connect with or otherwise communicate with one or more remote computers, such as a remote computer 944, which may be a personal computer, a server, a router, a network personal computer, a peer device, or other common network nodes, and typically includes many or all of the components described above in connection with the computer 900. Using appropriate communication devices 940, e.g., a modem, a network interface or adapter, etc., the computer 900 may operate within and communicate through a local area network (LAN) and a wide area network (WAN), but may also include other networks such as a virtual private network (VPN), an office network, an enterprise network, an intranet, the Internet, etc. It will be appreciated that the network connections shown are exemplary and other means of establishing a communications link between the computers 900, 944 may be used.

As used herein, the computer 900 includes or is operable to execute appropriate custom-designed or conventional software to perform and implement the processing steps of the method and system of the present invention, thereby forming a specialized and particular computing system. Accordingly, the presently-invented method and system may include one or more computers 900 or similar computing devices having a computer-readable storage medium capable of storing computer-readable program code or instructions that cause the processing unit 904 to execute, configure, or otherwise implement the methods, processes, and transformational data manipulations discussed hereinafter in connection with the present invention. Still further, the computer 900 may be in the form of a smartphone, a tablet computer, a personal computer, a personal digital assistant, a portable computer, a laptop, a palmtop, a mobile device, a mobile telephone, a server, or any other type of computing device having the necessary processing hardware to appropriately process data to effectively implement the presently-invented computer-implemented method and system.

Computer 944 represents one or more work stations appearing outside the local network and user and patient machines. The users and patients interact with computer 900, which can be an exchange system of logically integrated components including a database server and web server. In addition, secure exchange can take place through the Internet using secure www. An e-mail server can reside on system computer 900 or a component thereof. Electronic data interchanges can be transacted through networks connecting computer 900 and computer 944. Third party vendors represented by computer 944 can connect using any known protocol known to one skilled in the art to connect computers could be used.

The exchange system can be a typical web server running a process to respond to HTTP requests from remote browsers on computer 944. Through HTTP, the exchange system can provide the user interface graphics. It will be apparent to one skilled in the relevant art(s) that the system may utilize databases physically located on one or more computers which may or may not be the same as their respective servers. For example, programming software on computer 900 can control a database physically stored on a separate processor of the network or otherwise.

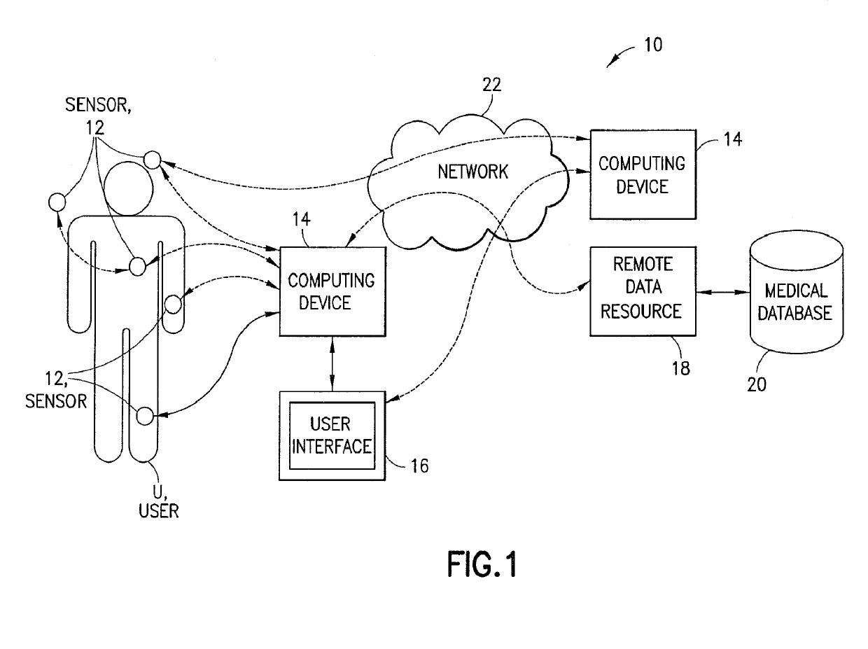

The present invention is directed to an automated personal medical diagnostic system and arrangement 10, which is illustrated in various preferred and non-limiting embodiments in FIGS. 1-9.

According to one preferred and non-limiting embodiment, and as illustrated in FIG. 1, provided is an automated personal medical measurement and diagnostic system 10. This system 10 includes at least one sensor 12 (and preferably multiple sensors 12) that are configured to measure and/or sense at least one physiological condition, and generate or acquire sensor data. It is further understood that these sensors 12 may be attached to a user U, which may be in the form of a patient P and/or a person whose measurements are taken and for whom a diagnosis, analysis, or recommendation is to be generated, worn by the user U, associated with the user U, physically embedded in the user U, adjacent the user U, spaced from the user U, and the like. Accordingly, the user U (as referred to hereinafter) may be any user, patient P, or any other person using or involved with the system 10. Further, it is also envisioned that the sensors 12 may be used to measure and/or sense additional information or data, such as environmental data, local data, or other data or information that can be used in the diagnostic process (as discussed hereinafter). Still further, it is envisioned that two or more sensors 12 may be in hard-wired or wireless communication with each other and transmit some or all of the sensor data therebetween.

The system 10 further includes at least one computing device 14 configured to process at least a portion of the sensor data and generate diagnostic data based at least partially on some or all of the sensor data. As discussed hereinafter, some or all of the diagnostic data and/or the sensor data can be displayed or graphically represented on a user interface 16, such as a user interface 16 associated with or integrated with the computing device 14. As discussed in more detail hereinafter, and in one preferred and non-limiting embodiment, computing device 14 is a portable mobile device (e.g., a smartphone or other mobile computing device), or, in another embodiment, a device with which a mobile device can be docked, stationed, or placed in communication. Further, the computing device may be attached to the user U, associated with the user U, worn by the user U, carried by the user U, remote from the user U, and the like. Still further, the sensors 12 may be included with, attached to, or integrated with the computing device 14, whether a mobile computing device, a local computing device, or a combination thereof. For example, as discussed hereinafter, the user interface 16 may be displayed on a first computing device 14 (e.g., a smartphone) and the processing to obtain the diagnostic data on a separate local computing device 14 and/or remote computing device 14. As discussed hereinafter, and in one preferred and non-limiting embodiment, the at least one computing device 14 may be in the form of one or more of the following: a local computing device, a remote computing device, a “cuff” device, a “sleeve” device, a “tabletop device”, an “enclosure” device, or any combination thereof.

Also, as seen in FIG. 1, the computing device 14 may be local to the user U (or patent P, if the user is the same as the person being measured/diagnosed, or for whom the recommendations are intended) with the user interface 16 integrated with the computing device 14 (e.g., a smartphone, a device in communication with the smartphone, etc.), and/or the computing device 14 may be remote from the user U and in communication with a user interface 16 (e.g., a server in communication with a display of a local computing device 14, e.g., a smartphone, a device in communication with the smartphone, etc.). In addition, the system 10 may include both a local computing device 14 (or multiple local computing devices 14) and a remote computing device 14 that are in communication with each other, and each device 14 may receive, process, and/or transmit some or all of the sensor data and/or the diagnostic data. Accordingly, the location of the various software modules and/or data storage may be chosen to suit any particular situation or environment. It is envisioned that one or more of the sensors 12 can be associated with, integrated with, contained within, or part of the at least one computing device 14.

In one preferred and non-limiting embodiment, and as discussed in greater detail hereinafter, the diagnostic data at least partially includes indicator data, medical diagnostic data, trigger data, or any combination thereof. As discussed, some or all of this diagnostic data (or some or all of the sensor data) can be displayed, in a variety of forms, on the user interface 16. For example, the data may be provided in alphanumeric textual form, graphical form, as a graphical representation, in report form, or in any other organized manner. Specifically, and in another preferred and non-limiting embodiment, some or all of the data and information is provided to the user U in a format that can be plainly and easily understood. Further, and as discussed hereinafter, some or all of the sensor data and/or the diagnostic data can be used to implement and/or initiate various other actions and sequences in the system 10. It is to be understood that, as used herein, the plural or singular terms or phrases “diagnosis”, “diagnostic”, “medical diagnosis”, “medical diagnostic” refer to all aspects (e.g., identification, determination, analysis, recommendation, reporting, etc.) of any medical, health, and/or wellness condition or issue, and data or information relating thereto.

Still further, and in another preferred and non-limiting embodiment, the user interface 16 includes a display for providing graphical information to the user U (whether the patient or some other person), and this graphical information is displayed dynamically, periodically, and/or on a predetermined or requested basis. This graphical information may represent at least a portion of at least one of the following: the measured or sensed data, the diagnostic data, the indicator data, the medical diagnostic data, the trigger data, the sensor data, input data, remote data resource data, medical record data, health record data, a personal health record, an electronic health record, a preexisting medical measurement, laboratory results, magnetic resonance imaging (MRI) scan data, visualization scan data, medical consultation data, preexisting sensor data, preexisting diagnostic data, biomedical data, user interaction data, medical indicator data, physiological parameter data, heart rate, facial expression data, protein data, body mass index, a condition, a potential condition, an elimination of a potential condition, an analysis, a severity of a condition, a severity of a potential condition, a relevancy of a condition, a recommendation, a recommendation for further analysis, a recommendation for further monitoring, a recommendation for medical consultation, a recommendation for tele-medical consultation, a recommendation for an in-person consultation, a recommendation for urgent care, a recommendation for emergency care, a recommendation for further assistance, a recommendation including further information, a recommendation including health data, a recommendation including wellness data, a reference to a current measurement, a reference to medical diagnostic data, an initiation of a process for subsequent diagnosis, an initiation of a process for subsequent measurement, a request to at least one user for input, an actuator action, a request to interact with the at least one computing device, a request to interact with the at least one sensor, alarm data, or any combination thereof.

As discussed, the computing device 14 (which may include multiple devices in communication in a hard-wired or wireless format) may include at least one of the following: a mobile wireless device, a smartphone, a mobile computing device, a wireless device, a hard-wired device, a network device, a docking device, a personal computer, a laptop computer, a pad computer, a personal digital assistant, a wearable device, a remote computing device, a server, a functional computing device, or any combination thereof. While, in one preferred and non-limiting embodiment, the primary computing device 14 is a smartphone (which may include the appropriate hardware and software components to implement the various described functions), it is also envisioned that the computing device 14 be any suitable computing device configured, programmed, or adapted to perform one or more of the functions of the described system 10.

Similarly, the sensor 12 may be in a variety of forms and structures suitable to measuring and/or sensing physiological information and/or other information (e.g., environmental information, local information, and the like). For example, the sensor 12 may be at least one of the following: a wireless sensor, a hard-wired sensor, a sensor device attached to a computing device, a sensor device integrated with a computing device, a sensor software module on a computing device, a sensor array, a controllable sensor, an analog sensor, a digital sensor, an embedded sensor, a pressure sensor, a light sensor, a visible light sensor, a far infrared sensor, a near infrared sensor, an image sensor, an ultraviolet light sensor, an acoustic sensor, a chemical sensor, a biological sensor, a biochemical sensor, an electrode, an electrical activity sensor, a magnetometer, or any combination thereof. Accordingly, in one preferred and non-limiting embodiment, some or all of the sensors 12 are in wireless or hard-wired communication with the computing device 14, which receives the raw, pre-processed, or processed sensor data and determines at least a portion of the diagnostic data. In a further preferred and non-limiting embodiment, the sensor 12 is configured for use during sedentary or non-sedentary activities, conscious or unconscious conditions, sleep states, and/or any determinable mental state.

In another preferred and non-limiting embodiment, the sensor 12 is in the form of at least one of the following: a light intensity sensor, a heart rate sensor, an imaging sensor, a facial image sensor, a protein data sensor, a breathing rate sensor, a temperature sensor, photoplethysmograph (PPG) data sensor, an electrocardiogram (ECG/EKG) data sensor, a head-worn electrocardiogram data sensor, a chest-worn electrocardiogram sensor, a pulse transit time sensor, a blood pressure sensor, an oxygen saturation (SpO2) data sensor, a pulse oximetry data sensor, or any combination thereof. In another preferred and non-limiting embodiment, multiple sensors 12 are used, such as multiple sensors 12 associated with the user U or the computing device 14, where the computing device 14 is configured, programmed, or configured to substantially simultaneously receive and/or process the sensor data from the sensors 12. In another preferred and non-limiting embodiment, the sensor 12 is configured to substantially simultaneously measure and/or sense multiple physiological conditions. In another preferred and non-limiting embodiment, the sensor 12 is a user-initiated or user-activated sensor.

With continued reference to FIG. 1, and in another preferred and non-limiting embodiment, the computing device 14 is in communication (preferably wireless communication over a network 22) with a remote data resource 18. In one preferred and non-limiting embodiment, the computing device 14 is configured, programmed, or adapted to receive input data from the remote data resource 18. For example, this remote data resource 18 may be in the form of a medical database 20, which includes or is populated with medical and/or health records, such as medical and/or health records relating to the user U (or patient). In one preferred and non-limiting embodiment, the medical and/or health records at least partially include at least one of the following: a personal health record, an electronic health record, a preexisting medical measurement, laboratory results, magnetic resonance imaging scan data, visualization scan data, medical consultation data, preexisting sensor data, preexisting diagnostic data, biomedical data, or any combination thereof. In another embodiment, at least a portion of the sensor data and/or diagnostic data is transmitted to the remote data resource 18 for inclusion in at least one medical and/or health record. Information and data from this remote data resource 18 can be used (together with at least a portion of the sensor data) to determine or create the diagnostic data. This will provide a more complete and comprehensive analysis and/or diagnosis for presentation or communication to the user U.

As discussed, the diagnostic data may take a variety of forms. Accordingly, and in another preferred and non-limiting embodiment, the diagnostic data includes or is at least partially based on user data input (such as input at the user interface 16), user interaction with the computing device 14 (such as user interaction with his or her smartphone), user interaction with a sensor 12 (such as user interaction with an actuatable or controllable function of the sensor 12, e.g., adjustment, turn “on”, turn “off”, etc.), and the like. Further, at least a portion of the diagnostic data may be generated based upon at least a portion of the sensor data and at least a portion of input provided by the user U.

As also discussed, the diagnostic data may at least partially be in the form of indicator data, which may include at least one of the following: medical indicator data, physiological parameter data, heart rate, facial expression data, protein data, body mass index, breathing rate, temperature, surface temperature, internal temperature, photoplethysmograph data, electrocardiogram data, pulse transit time, blood pressure, oxygen saturation data, pulse oximetry data, pain data, microfluidic data, acoustic data, imaging data, a microfluidic sensor, or any combination thereof. This indicator data may be indicative of a likely or potential medical, health, and/or wellness condition or issue (or indicative of an impending medical, health, and/or wellness condition or issue), which requires subsequent action (as discussed hereinafter).

In a further preferred and non-limiting embodiment, the medical diagnostic data includes at least one of the following: a condition, a potential condition, an elimination of a potential condition, an analysis, a severity of a condition, a severity of a potential condition, a relevancy of a condition, a recommendation, medical data, health data, wellness data, or any combination thereof. Accordingly, the medical diagnostic data can be created and provided to user U (or to a health professional or other person) for the informational purposes of the user U, as well as to present an action plan/recommendation. For example, the recommendation may include at least one of the following: a recommendation for further analysis, a recommendation for further monitoring, a recommendation for medical consultation, a recommendation for tele-medical consultation, a recommendation for an in-person consultation, a recommendation for urgent care, a recommendation for emergency care, a recommendation for further assistance, a recommendation including further information, a recommendation including health data, a recommendation including wellness data, or any combination thereof. Therefore, the user U is provided with a concrete plan for the further handling or management of the actual or potential medical, health, and/or wellness issue.

It is envisioned that the diagnostic data, sensor data, and/or recommendation may be associated with a timing issue or a need for urgent care. Accordingly, and in another preferred and non-limiting embodiment, the diagnostic data, sensor data, and/or recommendation data can be associated with at least one of the following: an alarm, an audible alarm, a visual alarm, a tactile alarm, a message, an audible message, a visual message, a tactile message, an indicator that attracts a user's attention, or any combination thereof. This will ensure that the user U is aware that some action should be taken. It is further envisioned that the message or alarm will be transmitted to a hospital, doctor's office, or other health services location.

In a still further preferred and non-limiting embodiment, the trigger data at least partially includes at least one of the following: a reference to a current measurement, a reference to medical diagnostic data, an initiation of a process for subsequent diagnosis, an initiation of a process for subsequent measurement, a request to at least one user for input, an actuator action, a request to interact with the at least one computing device, a request to interact with the sensor 12, or any combination thereof.

In another preferred and non-limiting embodiment, provided is a method for automated medical diagnosis, including: associating with at least one user U at least one sensor 12 configured to measure and/or sense at least one physiological condition and generate sensor data; providing at least one computing device 14 configured, programmed, or adapted to process at least a portion of the sensor data and generate diagnostic data based at least partially on the sensor data; and providing at least one user interface 16 configured for user interaction. In this embodiment, the diagnostic data at least partially includes at least one of the following: a condition, a potential condition, an elimination of a potential condition, an analysis, a severity of a condition, a severity of a potential condition, a relevancy of a condition, a recommendation, a recommendation for further analysis, a recommendation for further monitoring, a recommendation for medical consultation, a recommendation for tele-medical consultation, a recommendation for an in-person consultation, a recommendation for urgent care, a recommendation for emergency care, a recommendation for further assistance, a recommendation including further information, a recommendation including health data, a recommendation including wellness data, or any combination thereof.

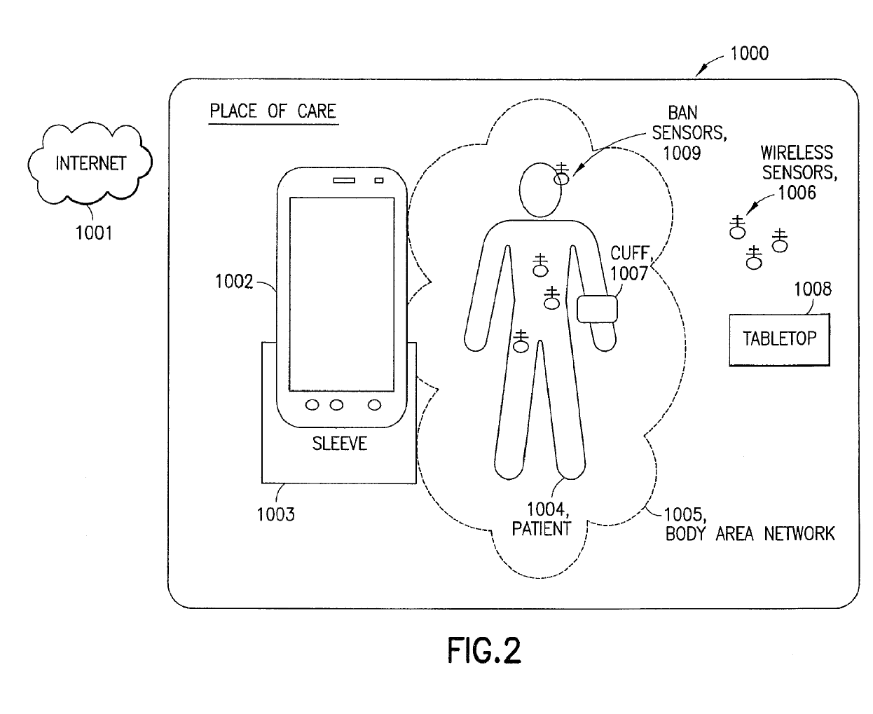

A further preferred and non-limiting embodiment of an automated personal medical measurement and diagnostic system and arrangement is illustrated in FIG. 2. In particular, and in this embodiment, the system is used at a place of care 1000. The system may employ one or more devices, which may take any form. In this embodiment, the system utilizes a mobile phone attachment 1003, referred to as a “sleeve” device in the remainder of this description of this embodiment. In another embodiment, a standalone device 1007, referred to as a “cuff” device in the remainder of this description of this embodiment, may be attached to or worn by a patient being diagnosed 1004. In yet another preferred and non-limiting embodiment, a stationary device 1008, referred to as a “tabletop” device in the remainder of this description of this embodiment, may be used. The system may use one or a combination of two, three, or more of the devices 1003, 1007, and 1008. For example, a sleeve device and a tabletop device may be used in combination.

Preferably, the devices may be used with an off-the-shelf smartphone 1002 using a wired connection and/or a wireless connection for connecting the devices with the smartphone 1002. Alternatively, any mobile or network device may be used to implement some or all of the medical diagnostic functionality of the system. Such devices may include computers, mobile devices, such as personal digital assistants (PDAs), including Palm-based or Windows CE devices, phones such as cellular phones and smartphones (e.g., iPhone, Blackberry, Android, Treo), any device capable of communicating wirelessly with a communications network and/or any other type of network device. Any description of smartphones herein may also be applied to any other mobile or network devices. Further, the system may also employ one or more wired and/or stationary devices.

With continued reference to FIG. 2, the devices 1003, 1007, and 1008 may each include or integrate a variety of sensors and wireless network interfaces. Readings obtained through these sensors may be transmitted via the wireless network interfaces to the sleeve 1003. Alternatively, sensor readings may also be transmitted directly to the smartphone 1002 (e.g., via Bluetooth data exchange) and/or to one or more other devices (e.g., from a tabletop device 1008 to a cuff device 1007), wherein the sensor readings may be further transferred from the one or more other devices to the smartphone 1002 directly and/or via a sleeve device 1003.

Additional wireless sensor modules 1006 and/or 1009 may also be used at the place of care. The sensors may be located inside and/or outside of a patient's body area network 1005, and may be physically attached to or worn by the user, or alternatively, spaced from the user. Readings from these modules may be transmitted to the sleeve 1003, the smartphone 1002, another device in the system and/or directly to the Internet 1001. In one preferred and non-limiting embodiment, an application running on the smartphone 1002 provides a user interface for the diagnostic process, and may communicate with health records and diagnostic services on the Internet 1001. Alternatively, one or more user interfaces may be provided through other devices, such as for example the cuff 1007 or tabletop 1008 devices described herein. These interfaces may provide additional functionality to that provided on the smartphone 1002. The interfaces may also replace part or all of the functionality provided on the smartphone 1002. Alternatively, these interfaces may complement the user interface provided on the smartphone 1002.

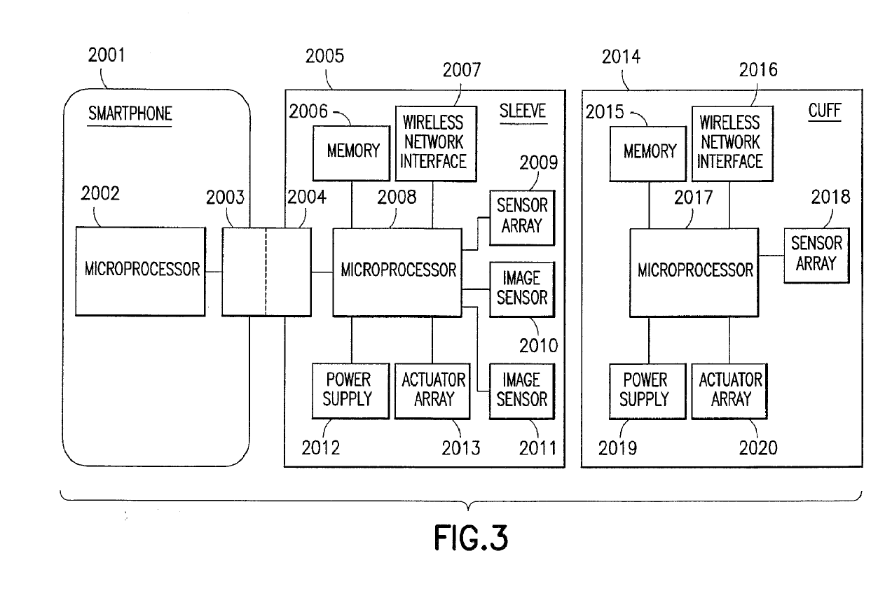

In another preferred and non-limiting embodiment, and as illustrated in FIG. 3, the system includes certain hardware components, such as those components used in connection with the sleeve device 1003 and/or the cuff device 1007. In a preferable embodiment, a sleeve 2005 may be attached through a USB host interface 2004 to a built-in USB client interface 2003 of an off-the-shelf smartphone 2001. USB interfaces 2003 and 2004 may be physically embodied as USB microconnectors, USB mini connectors, proprietary connectors aggregating USB signals with other signals, or any similar connectors. A cuff 2014 may not use a wired connection to the smartphone 2001 or the sleeve 2005, but may instead rely on one or more wireless network interfaces 2016 to communicate with a sleeve 2005 equipped with a wireless network interface 2007. In some embodiments, the sleeve 2005 may be attached to the smartphone 2001 through other connection arrangements employing wired or wireless data connection. Further, the cuff 2014 may use a wired connection to the smartphone 2001, the sleeve 2005, and/or other devices in the system. Alternatively, the cuff 2014 may rely on one or more wireless network interfaces 2016 to establish wireless communication directly with the smartphone 2001.

In another preferred and non-limiting embodiment, the sleeve 2005 and the cuff 2014 devices each include hardware components to allow execution of software instructions. Any description of these devices herein may also be applied to devices not including all or some of the hardware components shown in FIG. 3. For example, the devices may each include all, a subset of, or none of the components shown in FIG. 3. Further, each of the devices may include other components not shown in FIG. 3. Still further, any description of a sleeve device may also apply to a cuff device, and vice versa. A tabletop device may include any components of the sleeve and/or cuff devices and/or additional components.

The devices may be built around one or more microprocessors 2008 and 2017. Each microprocessor may be an 8-bit microcontroller, such as an Atmel ATmega168 AVR microcontroller, an integrated 32-bit system-on-chip, such as a Freescale i.MX35, or any other suitable microprocessor type. The devices may be equipped with volatile and non-volatile information storage components as discrete components (e.g., SDRAM chips, flash memory chips, SD memory cards), embedded within the microprocessors 2008 and 2017 or a combination thereof. Power supply circuitry 2012 and 2019 may provide operating power to the microprocessors, as well as other electrical components of the devices 2005 and 2014. The operating power may be provided by an internal or external power source. An internal power source may include any primary or secondary electrochemical or other storage device, such as a battery, a capacitor, a flywheel or any variation, combination, or specific incarnation thereof. Power may also be provided by external AC and/or DC power sources, through the USB interface 2004, and/or other means. Additional electronic circuitry may also be included, as specified by the specifications of the particular microprocessors 2008 and 2017. Such circuitry may include but is not limited to crystals and reference clocks, capacitors, pull-up and pull-down resistors for bus interfaces, level convertors, and line terminators.

With continued reference to FIG. 3, the devices 2005 and 2014 include a wide variety of sensors in sensor arrays 2009 and 2018, a variety of wireless network interfaces 2007 and 2016, a variety of actuators 2013 and 2020 (e.g., lens control for image sensors, IR and visible light sources, electrical outputs on electrodes) and/or memory 2006 and 2015 connected in various ways to the microprocessors 2008 and 2017. The sleeve device 2005 may include one, two or more image sensors 2010 and 2011, preferably attached directly to a dedicated image sensors interface of the microprocessor 2008. In some embodiments, such image sensors may be connected through other arrangements, such as a USB or SDIO bus, or by connection to a generic system bus of the microprocessor 2008. Further, image sensors may reside within other devices in the system and/or within the smartphone 2001.

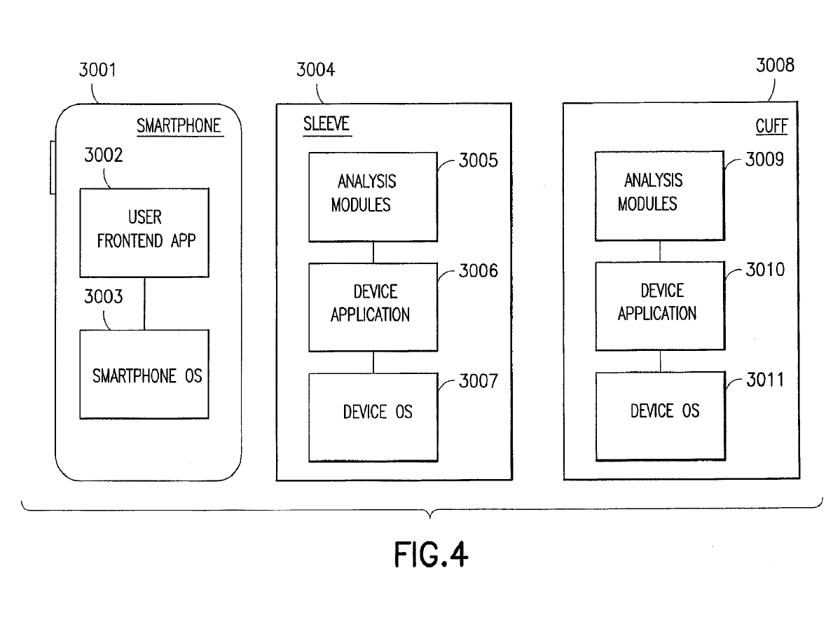

In another preferred and non-limiting embodiment, and as illustrated in FIG. 4, the system utilizes various software components. An off-the-shelf smartphone 3001 may be equipped with an operating system 3003 (such as Apple iOS in the case of an Apple iPhone smartphone, or a variation of Microsoft Windows Mobile or Windows Phone as, or a variant of Google Android), which may provide the capability to run third party applications on the smartphone. The system of the present invention may also provide implementations of a user front-end application 3002 running on the smartphone 3001, which may interact with network services on the Internet 1001 and/or hardware components of the system, such as a sleeve 3004 and/or a cuff 3008. The user front-end application 3002 may interact with the smartphone OS 3003 in order to provide an interactive graphical user interface to the operator of the diagnostic process, and may also rely on the 3003 smartphone OS for networking services with the Internet 1001 and/or for communication with the devices 3004 and 3008. In another embodiment, the system may utilize an object-based representation of the internal software processes to allow for modification of workflow processes after the software is compiled and installed.

The devices 3004 and 3008 may be equipped with a device as 3007 and 3011, which may be a variant of Google's Android platform, but may also be exemplified by a set of device driver and hardware abstraction modules. The device as 3007 and 3011 may provide system initialization code, hardware device component interface functionality and abstraction layers, file system interface code, as well as facilities to allow execution of one or more application modules. In other embodiments, the smartphone and/or the one or more devices described herein may not be equipped with an operating system. For example, the smartphone and/or the devices may be connected to a cloud computing or communication network. In such a configuration, all or some of the communication and operation of system components may take place via the cloud rather than locally.

A device application 3006 or 3010 may run within the devices 3004 and 3008. The device applications may aggregate sensor readings from sensors embedded in the devices and/or external wireless sensors. Further, the device applications may feed those readings, in addition to information derived from Internet-based services, into analysis modules 3005 and 3009. Results from modules 3005 and 3009 may be aggregated and used as input to the user interface application 3002. The results may also be used as additional input for further steps in the diagnostic process.

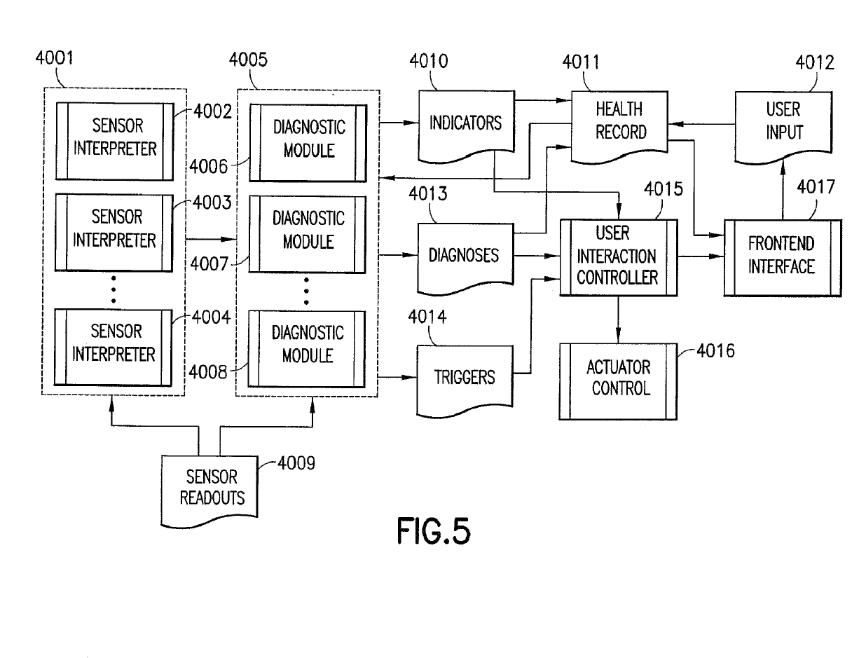

In another preferred and non-limiting embodiment, and as illustrated in FIG. 5, the system includes certain software modules and data flows of a device application. In this embodiment, the software modules may communicate with each other in the various ways indicated by the arrows representing data flows. One or more readouts 4009 from the various sensors embedded in the device or the external wireless sensors 1006 and 1009 may be interpreted by a variety of sensor interpretation modules 4001 to obtain medical indicators and readings of physiological parameters. For example, light intensity readouts from a light sensor may be continuously analyzed by a sensor interpretation module 4002 to calculate heart rate as a physiological parameter. In another example, facial image recognition routines in a sensor interpretation module 4003 may analyze facial expressions. In yet another example, sensor interpretation module 4004 may analyze signals from a protein analysis chip.

Interpreted sensor data from the interpretation modules 4001, the raw sensor readouts 4009, and/or historical measurements from a health record 4011 may be used as input for a variety of diagnostic modules 4005, which may include individual diagnostic modules 4006, 4007 and 4008. These diagnostic modules may generate medical and/or physiological indicators 4010 (e.g., body mass index (BMI)), medical diagnoses 4013 for one or more medical, health, and/or wellness conditions (both negative and positive) and triggers 4014 for subsequent steps in the diagnostic process. The health record 4011 may be an electronic record stored within or on one or more devices of the system. The device application software 3006 or 3010 may augment and synchronize this record with personal health records or electronic health records made available through Internet services 1001. This would allow diagnostic modules to make use of readouts from medical measurements taken outside of the place of care or through other facilities. Examples of such external measurements may include but are not limited to lab results, magnetic resonance imaging scans, reports from medical consultations, historical sensor readings, and/or diagnoses.

Some or all of the data may be stored on a local information storage medium, such as a hard disk. Alternatively, parts or all of the health record 4011 may be stored remotely. For example, the health record 4011 may be stored in a cloud database or in a data repository accessible via a communications network.

The diagnoses 4013 from the various diagnostic modules 4005 may each describe a particular medical, health, and/or wellness condition, the likelihood of that medical, health, and/or wellness condition being present in the patient, the likelihood of that medical, health, and/or wellness condition not being present in the patient, rationales for this analysis and an optional indicator for the severity of the medical, health, and/or wellness condition. Triggers 4014 may include references to a diagnostic module, a user interface action (for example, a question to be asked to the user or operator), an actuator action and/or an optional indicator of the importance of a subsequent diagnostic step to be taken. The various diagnoses 4013 and triggers 4014 may be aggregated by a user interaction controller 4015 to provide a summarized partial differential diagnosis and a list of subsequent steps, indexed by probability and severity. For a subset of the most relevant diagnoses, indicators may be sent to a front-end interface 4017. The front-end interface 4017 may interact with the user front-end application 3002 running on the smartphone 3001. The triggers 4014 may cause the user interaction controller 4015 to request actuator actions (such as turning on an infrared light source) from an actuator control module 4016 for use in subsequent diagnostic steps. The front-end interface module 4017 may send summarized results of the partial diagnosis as relevant biomedical indicators from the health record to the front-end application 3002. User input 4012 obtained through the smartphone application 3002 may also be processed by the front-end interface 4017 for inclusion in the health record 4011, where this user input may then be used as input for the diagnostic modules 4005.

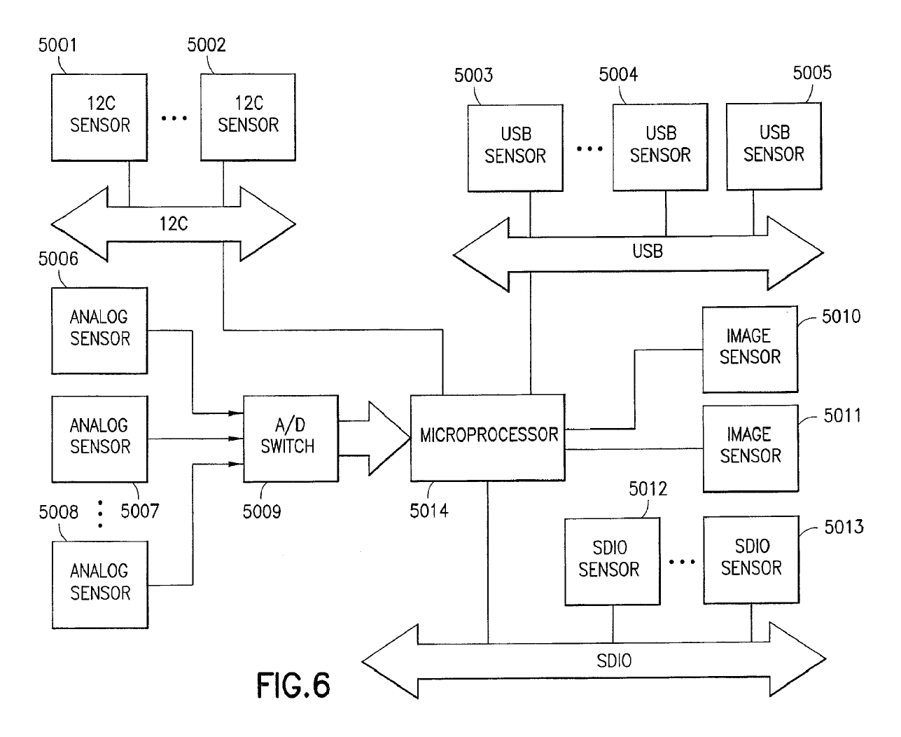

In a further preferred and non-limiting embodiment, and as illustrated in FIG. 6, the system uses certain hardware connections and sensors in implementation. For example, the system of the present invention is configured for use in connection with a wide variety of sensors to be embedded in one or more of the described devices. These may include, but are not limited to, pressure sensors, light sensors (both in the visible light spectrum, as well as image sensors for far and near infrared, ultraviolet and other frequency ranges of the electromagnetic spectrum), acoustic sensors, chemical and biological sensors, electrodes detecting electric activity, magnetometers and other sensors. Sensors embedded in a sleeve and/or cuff device may be connected to a microprocessor 5014 in a variety of ways. For example, sensors 5001 and 5002 with an I2C or SMBus interface may be connected to the microprocessor 5014 using an I2C bus. In another example, sensors 5003, 5004 and 5005 with a logical or physical USB interface may be connected with the microprocessor 5014 through an internal USB bus. Image sensors 5010 and 5011 may be connected directly to a dedicated image sensor interface of the microprocessor 5014. Some sensors may also have an SDIO interface, such as the SDIO sensors 5012 and 5013, which may be connected to the microprocessor 5014 using one or more SDIO busses. A variety of analog sensors 5006, 5007 and 5008 may be attached to a microprocessor's analog-to-digital interface through a signal matrix switch 5009 operated by the microprocessor, allowing more analog sensors to be multiplexed over a limited number of analog-to-digital interfaces of the microprocessor.

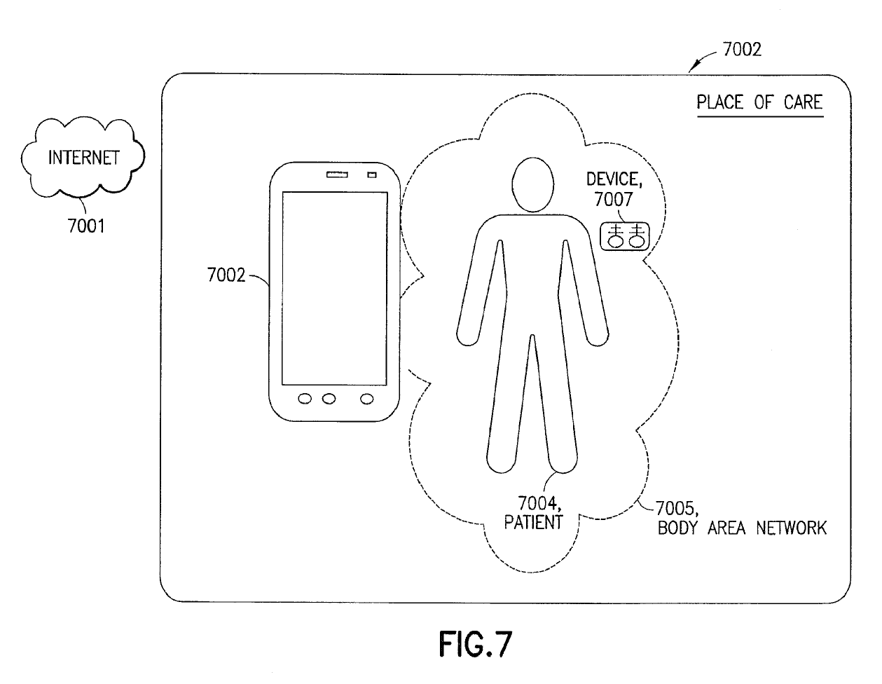

In a still further preferred and non-limiting embodiment, and as illustrated in FIG. 7 (and as similar to the embodiment of FIG. 2), the at least one computing device 14 includes a smartphone 7002 that is in communication with local computing device 7007. In this embodiment, the smartphone 7002 and/or the local computing device 7007 are associated with the patient 7004 and collectively or individually create a body area network 7005 at the place of care 7002 (e.g., the patient 7004 home, a hospital, a clinic, a health services location, and the like). Further, in this embodiment, the sensors are positioned on or integrated with the local computing device 7007, and any of the sensors, the local computing device 7007, and/or the smartphone 7002 are configured, programmed, or adapted to communicate over the Internet 7001 (as discussed above in greater detail). Accordingly, this embodiment demonstrates the use of two computing devices, i.e., the smartphone 7002 and the local computing device 7007, either or both of which can be configured, programmed, or adapted to implement any of the above-described processing and functions, or include the user interface 16. Of course, in one preferred and non-limiting embodiment, the smartphone 7002 displays the user interface to the patient 7004 for presentation of information and data (e.g., sensor data, diagnostic data, local data, environmental data, and the like) and facilitates interaction with the local computing device 7007 and/or the sensors.

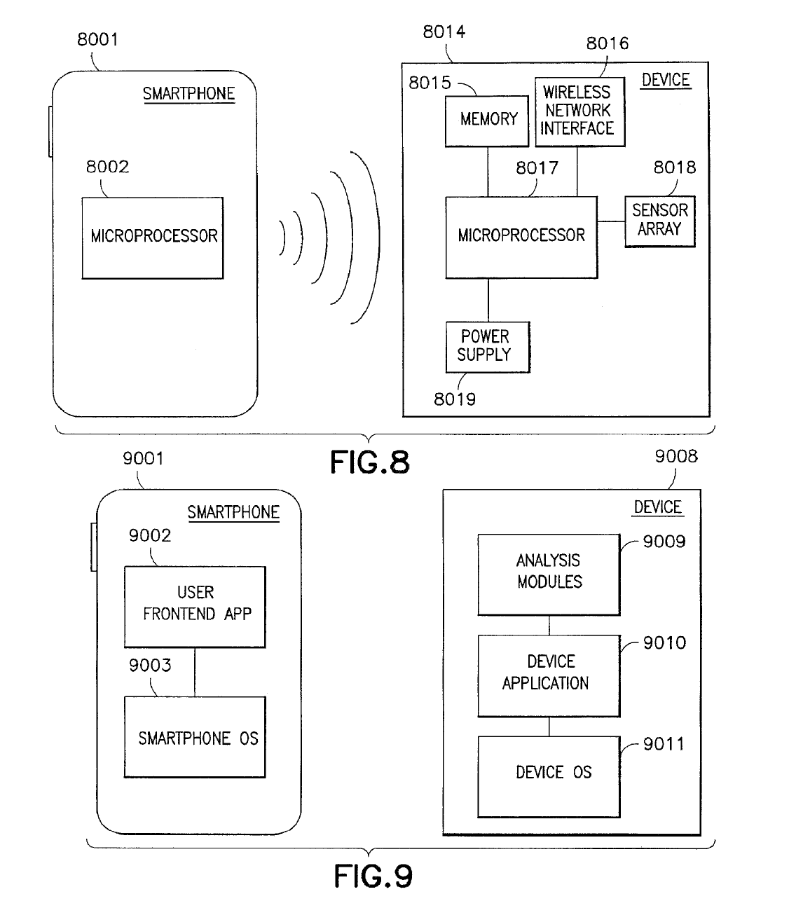

In a further preferred and non-limiting embodiment, and as illustrated in FIG. 8 (and as similar to the embodiment of FIG. 3), the smartphone 8001 includes a microprocessor 8002, as does the local computing device 8014. In this embodiment, the local computing device 8014 is similar in function to the sleeve device 2005 and/or the cuff device 2014 discussed above. Accordingly, the local computing device 8014 includes a memory 8015, a wireless network interface 8016, a microprocessor 8017, a sensor array 8018, and a power supply 8019. These components operate and function substantially as described above in connection with the preferred and non-limiting embodiment of FIG. 3. In addition, and in this embodiment, the smartphone 8001 is in wireless communication with the local computing device 8014. This facilitates fast communication between the sensors and/or the local computing device 8014, and the smartphone 8001.

In another preferred and non-limiting embodiment, and as illustrated in FIG. 9 (and as similar to the embodiment of FIG. 4), the smartphone 9001 includes a user front-end application 9002 and a smartphone operating system 9003. In addition, the local computing device 9008 includes the analysis modules 9009, the device application 9010, and the device operating system 9011. These software components operate and function substantially as described above in connection with the preferred and non-limiting embodiment of FIG. 4. Again, however, this embodiment utilizes a smartphone 9001 and a local computing device 9008, as opposed to the above-described sleeve device 3004 and cuff device 3008. The software modules and data flows of this embodiment operate and function substantially as described above in connection with the preferred and non-limiting embodiment of FIG. 5. Similarly, the hardware connections and sensor arrangement of this embodiment operate and function substantially as described above in connection with the preferred and non-limiting embodiment of FIG. 6.

In another preferred and non-limiting embodiment, the present invention provides systems, methods, and arrangements for acquiring, evaluating, screening, and presenting recommendation and/or diagnoses to a user, such as by initiation by the user U. Further, and in this embodiment, the system 10 allows for users, who are not medically trained, to acquire, evaluate, and present recommendations and/or diagnoses at user-initiated instances.

In another preferred and non-limiting embodiment, the at least one computing device 14 acts as or is in the form of an enclosure for one or more of the sensors 12 that are associated with the user U. For example, this enclosure may be a smart enclosure, i.e., including the necessary processing components to perform some or all of the functions discussed above. In this embodiment, the system 10 is implemented at a place of care for the user U, and the place of care may be any location where a user has access to the sensor enclosure, a smartphone, and the Internet. Accordingly, and in this embodiment, the at least one computing device 14 includes both the enclosure and the smartphone, which are in communication (preferably, wireless communication) with each other. The enclosure, which may be referred to as an “enclosure” device, may be in contact or associated with a user being diagnosed.

This “enclosure” device may operation, function, and/or act as or replace any one or more of the following: the local computing device, the remote computing device, the “cuff” device, the “sleeve” device, the “tabletop device”, or any combination thereof. Accordingly, the enclosure device may include a variety of sensors and wireless network interfaces, such that readings obtained through these sensors may be transmitted via the wireless network interfaces directly to the smartphone and/or to one or more other devices, where the sensor readings may be further transferred from the one or more other devices to the smartphone directly. One or more user interfaces 16 may be provided through the enclosure device. Further, the hardware components of the enclosure device may operate or function substantially similarly to that of any one or more of the computing device 14 (FIG. 1), device 1008 (FIG. 2), devices 2005, 2014 (FIG. 3), and device 8014 (FIG. 8), the software components of the enclosure device may operation or function substantially similarly to that of any one or more of the computing device (FIG. 1), device 1008 (FIG. 2), devices 3004, 3008 (FIG. 4), and device 9008 (FIG. 9), and the software modules, hardware connection, sensors, and data flows may operate or function substantially similarly to those illustrated in FIGS. 5 and 6.

A further aspect of the invention may provide a method for automated medical recommendation and/or diagnosis. The method may include providing a mobile medical diagnostic system in accordance with another aspect of the invention. One or more devices comprising a processing unit, an assortment of sensors and wireless network interfaces may be employed at the place of care to collect, process and communicate medical information and diagnosis with in an off-the-shelf smartphone.

Software running on the devices may record and process sensor data from embedded sensors, sensors in the smartphone and sensors connected through the devices' wireless network interfaces. The method may include using the sensor readings as inputs for a collection of diagnosis modules, which may yield estimations as to the likelihood of a particular medical, health, and/or wellness condition being present in the patient, as well as medical indicators and action triggers. These diagnostic module outputs may be aggregated in a user interaction module which may engage the user by providing a differential diagnosis and/or by prompting the user for additional information or instructions. The method may include graphical presentation of the medical recommendation and/or diagnosis to a user.

The invention may offer significant advantages with respect to existing options for medical recommendation and/or diagnosis. The systems and methods herein may be advantageously applied to enable medical recommendation and/or diagnosis at the place of care. With the advent of sophisticated sensors and advanced assay diagnostics, the invention may enable the practical deployment and integration of these sensor and diagnostic technologies. Furthermore, the invention may enable communication with remote heath records, which may allow personalized medicine to be integrated with medical information systems used today. Such integration may enable significant reductions of health care costs.

Another benefit may be the predictive nature of the methods and systems provided herein. Through predictive diagnosis using the algorithms and medical information provided in accordance with the present invention, the present systems and methods may provide a path for predicting health issues. Practicing the present invention may help monitoring and predicting medical, health, and/or wellness conditions in the home, thereby providing preventive medicine strategies for individual users.

In another preferred and non-limiting embodiment, provided is a method for automated medical diagnosis. In this embodiment, the method includes providing a mobile medical diagnostic system (such as one or more of the embodiments of the system discussed above). One or more devices including a processing unit, an assortment of sensors, and wireless network interfaces may be employed at the place of care to collect, process, and communicate medical information and diagnosis with in an off-the-shelf smartphone. Software running on the devices may record and process sensor data from embedded sensors, sensors in the smartphone and sensors connected through the devices' wireless network interfaces.

The method further includes using the sensor readings as inputs for a collection of diagnosis modules, which may yield estimations as to the likelihood of a particular medical, health, and/or wellness condition being present in the patient, as well as medical indicators and action triggers. These diagnostic module outputs may be aggregated in a user interaction module which may engage the user by providing a differential diagnosis and/or by prompting the user for additional information or instructions. The method further provides a full or partial graphical presentation of the medical diagnosis to a user.

In another preferred and non-limiting embodiment, the local computing device 14 and/or the remote computing device 14 are loaded with or otherwise configured, programmed, or adapted to execute various processes in order to process the sensor data and/or determine some or all of the diagnostic data, whether in raw, pre-process, or processed form. For example, the diagnostic data may include software modules to interpret or process the incoming sensor data (or, alternatively, a programmable sensor may also engage in this processing) for use in determining the diagnostic data. Also, as discussed, and based at least partially on the raw, pre-processed, processed, and/or interpreted sensor data, the local computing device 14 and/or the remote computing device 14 determine some or all of the diagnostic data.

In one exemplary embodiment, the software modules that implement a “sensor interpretation” function (as based upon an algorithm and/or process) may include: Heartbeat detection from electrocardiogram signals; Heartbeat detection from photoplethysmograph signals; Heart rate detection from heartbeats; Pulse transit time from photoplethysmograph-derived heartbeats and electrocardiogram-derived heartbeats; and/or Blood oxygenation based on photoplethysmograph signals at two-light frequencies. In certain preferred and non-limiting embodiments, the “sensor interpretation” algorithms/processes are as follows:

Heartbeat Detection from Electrocardiogram Signals

As input, a sequence of samples from electrodes is used. These samples can be represented as a series of numbers, one for each sample, each corresponding to the electrode signal level at a given point in time.
1. Filter input data with a 10-30 Hz bandpass filter. Such a filter can be implemented as a software-implemented digital filter, for example using a fast Fourier transform.
   1.a. Convert the discrete-time input signal to its discrete Fourier transform (DFT) using a Fast Fourier Transform algorithm.
   1.b. In the DFT transform data, multiply the magnitude component of those frequency bins corresponding to frequencies outside of the desired range (10 Hz-30 Hz) with 0, and frequency bins within the desired range (10 Hz-30 Hz) with 1. In a more sophisticated variant, carefully chosen constant multiplication factors can be chosen to optimize the output of the filter.
   1.c. Convert the frequency-domain DFT data back to a time-domain series, by applying an inverse Fourier transform on the DFT data, yielding the filtered electrocardiogram data.
2. Calculate the arithmetic mean value of the filtered electrocardiogram data.
3. Set the variable “threshold” as the value of this arithmetic mean, multiplied by 1.5.
4. Iterate through the samples in the filtered DFT data, store the position of every value in the filtered electrocardiogram data which is higher than the threshold and for which the previous data point is lower than the threshold.
5. Result is a series of positions determined in step 4. Given the sample rate of the electrocardiogram input signal, these positions indicate the time-offset of each heartbeat.

Heartbeat Detection from Photoplethysmograph Signals

As input, a sequence of samples from a light detector (photo diode) is used. These samples can be represented as a series of numbers, one for each sample, each corresponding to the light intensity level at a given point in time.
1. Filter input data with a 10-30 Hz bandpass filter. Such a filter can be implemented as a software-implemented digital filter, for example using a fast Fourier transform:
   1.a. Convert the discrete-time input signal to its discrete Fourier transform (DFT) using a Fast Fourier Transform algorithm.
   1.b. In the DFT transform data, multiply the magnitude component of those frequency bins corresponding to frequencies outside of the desired range (10 Hz-30 Hz) with 0, and frequency bins within the desired range (10 Hz-30 Hz) with 1. In a more sophisticated variant, carefully chosen constant multiplication factors can be chosen to optimize the output of the filter.
   1.c. Convert the frequency-domain DFT data back to a time-domain series, by applying an inverse Fourier transform on the DFT data, yielding the filtered photoplethysmograph data.
2. Calculate the arithmetic mean value of the filtered electrocardiogram data. Set the variable “threshold” as the value of this arithmetic mean.
3. Iterate through the samples in the filtered photoplethysmograph data, store the position of every value in the filtered electrocardiogram data which is higher than the threshold and for which the previous data point is lower than or equal to the threshold.
4. Result is a series of positions determined in step 3. Given the sample rate of the electrocardiogram input signal, these positions indicate the time-offset of each heartbeat.

Heart Rate Detection from Heartbeats

As input, a sequence of heartbeat time positions is used (for example, as derived from the algorithms described above). Given a sequence of heartbeat time positions, with “lastPos” being the position of the last peak, and “firstPos” being the position of the first peak, “peakCount” being the number of peaks, and “sampleRate” being the sample rate of the input signal to which the positions correspond, average heart rate over the sequence of heartbeat time positions can be calculated using this formula: heartrate=sampleRate*(peakCount−1)/(lastPos−firstPos).

Pulse Transit Time from Photoplethysmograph-Derived Heartbeats and Electrocardiogram-Derived Heartbeats

As input, a sequence of heartbeat time positions derived from photoplethysmograph, and a sequence of heartbeat time positions derived from electrocardiogram. The algorithm assumes the time positions are identified by sample number, photoplethysmograph and electrocardiogram signals use an identical, known, sample rate.
1. Set “pairCounter” to 0.
2. Set “deltaSum” to 0.
3. For every entry in the electrocardiogram peak position sequence:
   3.a. Select entry in photoplethysmograph peak position array with the lowest position value that is greater than the position of the electrocardiogram peak being considered.
   3.b. If such a photoplethysmograph peak position is found:
      3.b.i Add the difference between the selected photoplethysmograph peak position and the electrocardiogram peak position to “deltaSum”.
      3.b.ii. Increase “pairCounter” by 1.
4. Result=deltaSum*sampleRate/pairCounter

This result is the average time between heartbeats as identified by analyzing electrocardiogram signals, and heartbeats as detected using photoplethysmograph. Since peaks are detected by photoplethysmograph sensors at the time when a blood pulse reaches the area of the body where the photoplethysmograph sensor is located, and electrocardiogram signals are detected almost instantaneous, pulse transit time correlates with the time it takes for a blood pulse to travel from the heart to the area near where the photoplethysmograph sensor is located. It should also be noted that this algorithm can equally be applied to calculate pulse transit time between two sequences of photoplethysmograph-derived heartbeats, if each of these sequences is based on simultaneous photoplethysmograph sensor readings at distinct positions on the body.

Blood Oxygenation Based on Photoplethysmograph Signals at Two-Light Frequencies

As input, a synchronized pair of photoplethysmograph sequences is used, each photoplethysmograph data at a different light frequency. The algorithm uses this data to calculate the oxygen saturation of oxygen saturation of hemoglobin in blood. In one type of photoplethysmograph/oxygen saturation sensor setup, this data is acquired using a common light sensor (such as a photo diode), and alternately illuminating a body part using one frequency and another. In another possible photoplethysmograph/oxygen saturation sensor setup, the body part is illuminated constantly, or at least during those periods when the signal from the light sensor is being sampled, but distinct light sensors are used, each having a color filter to only allow light at distinct frequency bands to be passed through.

In order to be able to estimate blood oxygenation, the distinct light frequencies used must be chosen such that light absorbance at each frequency differs significantly between oxygen-saturated and de-saturated hemoglobin. Typically, the frequencies used would be 660 nm (red) as the first light frequency, and 905, 910 or 940 nm as the second frequency.
1. Filter input data of both photoplethysmograph sample sequences with a 10-30 Hz bandpass filter. Such a filter can be implemented as a software-implemented digital filter, for example using a fast Fourier transform.
   1.a. Convert the discrete-time input signal to its discrete Fourier transform (DFT) using a Fast Fourier Transform algorithm.
   1.b. In the DFT transform data, multiply the magnitude component of those frequency bins corresponding to frequencies outside of the desired range (10 Hz-30 Hz) with 0, and frequency bins within the desired range (10 Hz-30 Hz) with 1. In a more sophisticated variant, carefully chosen constant multiplication factors can be chosen to optimize the output of the filter.
   1.c. Convert the frequency-domain DFT data back to a time-domain series, by applying an inverse Fourier transform on the DFT data, yielding the filtered photoplethysmograph data.
2. Calculate AC and DC components of each photoplethysmograph signal, based on filtered sequences from step 1.
   DCr:=mean(Sr)
   DCir:=mean(Sir)
   ACr:=max(Sr)−min(Sr)
   ACir:=max(Sir)−min(Sir)
3. normalizedRatio=(ACr/DCr)/(ACir/DCir).
4. result=constantA+(normalizedRatio*constantB). (constantA and constantB are calibration constants determined by emperically comparing the output of the system (software+hardware) with observed oxygen saturation values from a reference oxygen saturation measurement device).

Various of these algorithms and processes are based upon one or more of the following references (all of which are incorporated herein in their entirety): Pulse transit time: an appraisal of potential clinical applications, Thorax 1999; 54:452-457 [doi:10.1136/thx.54.5.452] [http://thorax.bmj.com/content/54/5/452.full]; U.S. Pat. No. 6,723,054; U.S. Pat. No. 6,527,728; U.S. Publication No. 2007/0276632; and U.S. Publication No. 2003/0199771; Severinghaus, John W., Honda Yoshiyuki (April 1987), “History of Blood Gas Analysis. VII. Pulse Oximetry”, Journal of Clinical Monitoring #(2): 135-138; Millikan G. A. (1942). “The oximeter: an instrument for measuring continuously oxygen-saturation of arterial blood in man”, Rev. Sci. Instrum 13 (10): 434-44 [doi:10.1063/1.1769941]; U.S. Pat. No. 6,385,471; U.S. Pat. No. 5,934,277; U.S. Pat. No. 5,503,148; U.S. Pat. No. 5,351,685; U.S. Pat. No. 5,219,381; U.S. Pat. No. 4,883,353; U.S. Pat. No. 4,824,242; U.S. Pat. No. 4,807,631; U.S. Pat. No. 4,796,636; U.S. Pat. No. 4,714,080; U.S. Pat. No. 4,623,248; and U.S. Pat. No. 4,266,554.

In one exemplary embodiment, the software modules that implement a “diagnostic” function (as based upon an algorithm and/or process) to provide diagnostic data may include: BMI calculation based on body height and body mass; fever detection; and prevalence-based symptom matching. In certain preferred and non-limiting embodiments, the “diagnostic” algorithms/processes are as follows:

BMI Calculation Based on Body Height and Body Mass

As input, body mass and height are extracted from a health record. Alternatively, one or more of these measurements may have been entered manually, or retrieved via a wired or wireless network connection with a digital weighing scale. The result is obtained by: mass (kg)/(height (m))̂2.

Fever Detection

Fever can be detected through a basic implementation or a more sophisticated implementation. A basic implementation of a fever detection algorithm compares body temperature with a fixed threshold value, as follows.
1. Health record available and containing body temperature entries?
   1.a. If yes: Tthreshold:=Average body temperature from health record
   1.b. If no: Tthreshold:=100 degrees Fahrenheit
2. If (Tbody>Tthreshold)
   Result:=true
   Else:
   Result:=false

A more sophisticated implementation of fever detection may utilize the following algorithm. Since “normal” body temperature varies during the day, and also varies from person to person, another way to detect fever is to use a comparison with a time-dependent threshold, as follows:
1. Health record available and containing body temperature entries?
   1.a. If yes: Threshold:=Average body temperature from health record for temperature measurements taken at the same time of day (within a specified time window, for example, 1 hour).
   1.b. If not: Threshold:=Expected body temperature at a given time of day (As taken from a static array of typical human body temperatures at a given time of day, for example as found in Encyclopedia Britannica, 11th edition, volume 2, part 1 http://www.gutenberg.org/files/13600/13600-h/13600-h.htm [v.02 p. 0049]).
2. If (Tbody>Threshold)
Result:=true
Else:
Result:=false

Prevalence-Based Symptom Matching

A set of symptoms are used, which may be manually entered, or results of a “sensor interpreter” or “diagnostic module” software modules. In this example, symptom indications are treated as binary: for example, “fever” (yes/no), “headache” (yes/no), “elevated blood pressure” (yes/no). These symptoms are compared with a database with, for every disease incorporated in this diagnostic module, including, for example: prevalence of the disease in a specific demographic (for example, in relation to the total U.S. population); and correlation factors of that disease with each symptom incorporated in the diagnostic module, for example, on a scale from 0 to 1. These correlation factors may be determined in a variety of manners, e.g., utilizing domain experts to set initial values, implement diagnostic system and modify the correlation factors until the outcome of the system matches expectations, etc. In one exemplary embodiment, the process/algorithm is as follows:
1. For every disease in the database:
   1.1. matchScore:=0
   1.2. For every symptom in the database:
      1.2.1. If the correlation factor for the disease is >constantA for the disease in the iteration:
         1.2.1.1. If the symptom is present in the symptom input set:
         matchScore:=matchScore+(symptomCorrelationFactor*constantB)+ConstantC;
         1.2.1.2. Else:
         matchScore:=matchScore−(symptomCorrelationFactor*constantD)−constantE;
      1.2.2. If the correlation factor for the disease is <=constantF for the disease in the iteration:
         1.2.2.1. If the symptom is present in the symptom input set:
         matchScore:=matchScore+(symptomCorrelationFactor*constantG)+constantH;
         1.2.2.2. Else:
         matchScore:=matchScore−(symptomCorrelationFactor*constantI)−constantJ;
      1.2.3. diseaseProbability:=matchScore*diseasePrevalence
      1.2.4. Add this “diseaseProbability”, together with a reference to the disease, to
         the result set of the algorithm.
2. Sort the result set in step 1 (containing a reference for each disease in the database, and “diseaseProbility” entry from steps 1.2.3 above) according to “diseaseProbability”.

The result of this algorithm is a list of diseases, ranked by probability, given the specified symptoms. Thresholds and constants can be chosen freely (but different choices here would affect the outcome, such that it is recommended that they are chosen empirically). A possible choice of constants includes:

constantA:=0.5
constantB:=1.0
constantC:=0.0
constantD:=0.0
constantE:=0.4
constantF:=0.5
constantG:=1.0
constantH:=0.0
constantI:=0.0
constantJ:=0.0

In this manner, the presently-invented systems, methods, and arrangements offer significant advantages with respect to existing options for medical diagnosis, which again, includes the identification, determination, analysis, recommendation, and/or reporting of any medical, health, and/or wellness condition or issue, and data or information relating thereto. The systems, methods, and arrangement described herein may be advantageously applied to enable medical diagnosis at any place of care. With the advent of sophisticated sensors and advanced assay diagnostics, the invention may enable the practical deployment and integration of these sensor and diagnostic technologies. Furthermore, the invention enables communication with remote heath records, which may allow personalized medicine to be integrated with medical information systems used today. Such integration enables significant reductions of health care costs. Further, the systems, methods, and arrangements of the present invention provide a beneficial predictive functionality. In particular, and through predictive diagnosis using the algorithms and medical information provided in accordance with the present invention, the present systems, and arrangements provide a path for predicting health issues. Further, the present invention facilitates the monitoring and prediction of medical, health, and/or wellness conditions in the home, thereby providing preventive medicine strategies for individual users.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. An automated personal medical diagnostic system, comprising:
at least one sensor configured to measure and/or sense at least one physiological condition and generate or acquire sensor data;
at least one computing device configured to process at least a portion of the sensor data and generate diagnostic data based at least partially on the sensor data; and
at least one user interface configured for user interaction;
wherein the diagnostic data at least partially comprises at least one of the following: indicator data, medical diagnostic data, trigger data, or any combination thereof.

2. The automated personal medical diagnostic system of claim 1, wherein at least one computing device comprises at least one of the following: a mobile wireless device, a smartphone, a mobile computing device, a wireless device, a hard-wired device, a network device, a docking device, a personal computer, a laptop computer, a pad computer, a personal digital assistant, a wearable device, a remote computing device, a server, a functional computing device, or any combination thereof.

3. The automated personal medical diagnostic system of claim 1, wherein the at least one sensor is at least one of the following: a wireless sensor, a hard-wired sensor, a sensor device attached to a computing device, a sensor device integrated with a computing device, a sensor software module on a computing device, a sensor array, a controllable sensor, an analog sensor, a digital sensor, an embedded sensor, a pressure sensor, a light sensor, a visible light sensor, a far infrared sensor, a near infrared sensor, an image sensor, an ultraviolet light sensor, an acoustic sensor, a chemical sensor, a biological sensor, a biochemical sensor, an electrode, an electrical activity sensor, a magnetometer, or any combination thereof.

4. The automated personal medical diagnostic system of claim 1, wherein the at least one computing device is configured to receive input data from at least one remote data resource over at least one network.

5. The automated personal medical diagnostic system of claim 4, wherein the remote data resource comprises at least one medical database comprising medical and/or health record data.

6. The automated personal medical diagnostic system of claim 5, wherein the medical and/or health record data comprises at least one of the following: a personal health record, an electronic health record, a preexisting medical measurement, laboratory results, magnetic resonance imaging scan data, visualization scan data, medical consultation data, preexisting sensor data, preexisting diagnostic data, biomedical data, or any combination thereof.

7. The automated personal medical diagnostic system of claim 1, wherein the diagnostic data is generated based at least partially on at least a portion of at least one of the following: user data input, user interaction with the at least one computing device, user interaction with the at least one sensor, or any combination thereof.

8. The automated personal medical diagnostic system of claim 1, wherein the indicator data comprises at least one of the following: medical indicator data, physiological parameter data, heart rate, facial expression data, protein data, body mass index, breathing rate, temperature, surface temperature, internal temperature, photoplethysmograph data, electrocardiogram data, pulse transit time, blood pressure, oxygen saturation data, pulse oximetry data, pain data, microfluidic data, acoustic data, imaging data, a microfluidic sensor, or any combination thereof.

9. The automated personal medical diagnostic system of claim 1, wherein the at least one sensor is at least one of the following: a light intensity sensor, a heart rate sensor, an imaging sensor, a facial image sensor, a protein data sensor, a breathing rate sensor, a temperature sensor, photoplethysmograph data sensor, an electrocardiogram data sensor, a head-worn electrocardiogram data sensor, a chest-worn electrocardiogram sensor, a pulse transit time sensor, a blood pressure sensor, a oxygen saturation data sensor, a pulse oximetry data sensor, or any combination thereof.

10. The automated personal medical diagnostic system of claim 1, wherein the medical diagnostic data comprises at least one of the following: a condition, a potential condition, an elimination of a potential condition, an analysis, a severity of a condition, a severity of a potential condition, a relevancy of a condition, a recommendation, medical data, health data, wellness data, or any combination thereof.

11. The automated personal medical diagnostic system of claim 10, wherein the recommendation comprises at least one of the following: a recommendation for further analysis, a recommendation for further monitoring, a recommendation for medical consultation, a recommendation for tele-medical consultation, a recommendation for an in-person consultation, a recommendation for urgent care, a recommendation for emergency care, a recommendation for further assistance, a recommendation including further information, a recommendation including health data, a recommendation including wellness data, or any combination thereof.

12. The automated personal medical diagnostic system of claim 11, wherein the at least one recommendation is associated with at least one of the following: an alarm, an audible alarm, a visual alarm, a tactile alarm, a message, an audible message, a visual message, a tactile message, an indicator that attracts a user's attention, or any combination thereof.

13. The automated personal medical diagnostic system of claim 1, wherein the trigger data comprises at least one of the following: a reference to a current measurement, a reference to medical diagnostic data, an initiation of a process for subsequent diagnosis, an initiation of a process for subsequent measurement, a request to at least one user for input, an actuator action, a request to interact with the at least one computing device, a request to interact with the at least one sensor, or any combination thereof.

14. The automated personal medical diagnostic system of claim 1, wherein at least a portion of at least one of the sensor data and the diagnostic data is transmitted to at least one remote data resource for inclusion in at least one medical and/or health record.

15. The automated personal medical diagnostic system of claim 1, further comprising a plurality of sensors, wherein the at least one computing device is configured to substantially simultaneously receive and/or process the sensor data from the plurality of sensors.

16. The automated personal medical diagnostic system of claim 1, wherein the at least one sensor is configured to substantially simultaneously measure and/or sense a plurality of physiological conditions.

17. The automated personal medical diagnostic system of claim 1, wherein the at least one user interface comprises a display for providing graphical information to at least user, wherein the graphical information is displayed dynamically, periodically, and/or on a predetermined or requested basis.

18. The automated personal medical diagnostic system of claim 17, wherein the graphical information represents at least a portion of at least one of the following: the measured or sensed data, the diagnostic data, the indicator data, the medical diagnostic data, the trigger data, the sensor data, input data, remote data resource data, medical record data, health record data, a personal health record, an electronic health record, a preexisting medical measurement, laboratory results, magnetic resonance imaging scan data, visualization scan data, medical consultation data, preexisting sensor data, preexisting diagnostic data, biomedical data, user interaction data, medical indicator data, physiological parameter data, heart rate, facial expression data, protein data, body mass index, a condition, a potential condition, an elimination of a potential condition, an analysis, a severity of a condition, a severity of a potential condition, a relevancy of a condition, a recommendation, a recommendation for further analysis, a recommendation for further monitoring, a recommendation for medical consultation, a recommendation for tele-medical consultation, a recommendation for an in-person consultation, a recommendation for urgent care, a recommendation for emergency care, a recommendation for further assistance, a recommendation including further information, a recommendation including health data, a recommendation including wellness data, a reference to a current measurement, a reference to medical diagnostic data, an initiation of a process for subsequent diagnosis, an initiation of a process for subsequent measurement, a request to at least one user for input, an actuator action, a request to interact with the at least one computing device, a request to interact with the at least one sensor, alarm data, or any combination thereof.

19. The automated personal medical diagnostic system of claim 1, wherein the at least one sensor is configured for use during at least one of the following: sedentary or non-sedentary activities, conscious or unconscious conditions, sleep states, mental states, or any combination thereof.

20. The automated personal medical diagnostic system of claim 1, wherein at least a portion of the diagnostic data is generated based upon at least a portion of the sensor data and at least a portion of input provided by at least one user.

21. The automated personal medical diagnostic system of claim 1, wherein the at least one sensor is a user-initiated or user-activated sensor.

22. The automated personal medical diagnostic system of claim 1, wherein at least a portion of at least one of the sensor data and the diagnostic data is associated with at least one of the following: an alarm, an audible alarm, a visual alarm, a tactile alarm, a message, an audible message, a visual message, a tactile message, an indicator that attracts a user's attention, or any combination thereof.

23. A method for automated medical diagnosis, comprising:
associating with at least one user at least one sensor configured to measure and/or sense at least one physiological condition and generate or acquire sensor data;
providing at least one computing device configured to process at least a portion of the sensor data and generate diagnostic data based at least partially on the sensor data; and
providing at least one user interface configured for user interaction;
wherein the diagnostic data at least partially comprises at least one of the following: a condition, a potential condition, an elimination of a potential condition, an analysis, a severity of a condition, a severity of a potential condition, a relevancy of a condition, a recommendation, a recommendation for further analysis, a recommendation for further monitoring, a recommendation for medical consultation, a recommendation for tele-medical consultation, a recommendation for an in-person consultation, a recommendation for urgent care, a recommendation for emergency care, a recommendation for further assistance, a recommendation including further information, a recommendation including health data, a recommendation including wellness data, or any combination thereof.
